(19) United States
(12) Patent Application Publication
(10) Pub. No.: US 2012/0087954 A1
(43) Pub. Date: Apr. 12, 2012

(54) ION SUBSTITUTED CALCIUM PHOSPHATE COATINGS

(75) Inventors: Wei Xia, Uppsala (SE); Carl Lindahl, Goteborg (SE); Hakan Engqvist, Osthammar (SE); Peter Thomsen, Vastra Frolunda (SE); Jukka Lausmaa, Goteborg (SE)

(73) Assignee: BIOMATCELL AB, Goteborg (SE)

(21) Appl. No.: 13/266,533

(22) PCT Filed: Apr. 26, 2010
(86) PCT No.: PCT/SE2010/050461

§ 371 Date: Dec. 16, 2011

(30) Foreign Application Priority Data
Apr. 27, 2009 (SE) 0900560-4

(51) Int. Cl. A61K 33/42; C23C 16/44; A61K 9/00; B05D 3/10; B05D 3/02; B05D 5/00; B05D 3/06

(52) U.S. Cl. 424/400; 427/2.24; 424/602

(57) ABSTRACT

A method for the formation of a surface coating of an ion substituted calcium phosphate on a substrate, the coating itself and the use of the coating.

### BACKGROUND OF THE INVENTION

The present invention relates to a method for the formation of a surface coating, specifically a crystalline surface coating, of an ion substituted calcium phosphate on a substrate. The invention also relates to a ion substituted calcium phosphate surface coating produced by the method.

Calcium phosphate in bone is a multi-substituted calcium phosphate, including traces of CO₃²⁻, F⁻, Cl⁻, Mg²⁺, Sr²⁺, Si⁴⁺, Zn²⁺, Ba²⁺, Fe³⁺, etc [1-3]. These ionic substitutions play an important role in bone formation and normal functions, such as the solubility and surface chemistry of the material.

Carbonate (CO₃²⁻) is the most abundant (2-8 wt %) anionic substitute, and partially substitutes both in the PO₄³⁻ site and the OH⁻ site of the calcium phosphate structure. The high reactivity of young bone could be related to the greater presence of carbonate compared with old bone. Carbonated calcium phosphate has showed improved solubility, collagen deposition in vitro and reabsorption in vivo, compared with stoichiometric calcium phosphate.

Fluoride exists in bone and teeth of vertebrate bodies. It was reported that the substitution of fluoride for OH sites and formation of fluoride-substituted hydroxyapatite enhanced the acid resistance and the mechanical properties of hydroxyapatite bioceramics [4], and induced better biological response [5]. Because of the superior acid resistance and the mechanical property, the fluoride-substituted hydroxyapatite is a beneficial coating on the dental implant.

Silicon has been found to be essential for normal bone and cartilage growth and development. Synthetic hydroxyapatite that includes trace levels of Si in its structure demonstrates markedly increased biological performance in comparison to stoichiometric hydroxyapatite [6]. The improvement in biological performance can be attributed to Si-induced changes in the material properties and also to the direct effects of Si in physiological processes of the bone and connective tissue systems. Si substitution promotes biological activity by the transformation of the material surface to a biologically equivalent hydroxyapatite by increasing the solubility of the material, by generating a more electronegative surface and by creating a finer microstructure. Release of Si complexes to the extracellular media and the presence of Si at the material surface may induce additional dose-dependent stimulatory effects on cells of the bone and cartilage tissue systems [6].

Because strontium is chemically and physically closely related to calcium, it is easily introduced as a natural substitution of calcium in hydroxyapatite. Strontium has proved to have the effects of increasing bone formation and reducing bone resorption, leading to a gain in bone mass and improved bone mechanical properties in normal animals and humans. Sr substituted hydroxyapatite ceramics have exhibited better mechanical properties than pure hydroxyapatite, and enhanced the proliferation and differentiation of osteoblast cells in in vitro study [7].

Magnesium has been found in high concentrations in bone and cartilage tissue during the initial phases of osteogenesis, and to cause the acceleration of the nucleation kinetics of hydroxyapatite and to inhibit its crystallization process. Landi et al. have observed that Mg substituted hydroxyapatite improved the behavior of cells in term of adhesion, proliferation and metabolic activity, as compared to stoichiometric hydroxyapatite [8].

Zinc is a major trace element in bone, and has been found to play a major role in human tissue development. The in vitro experiments have shown that zinc inhibits bone resorption, and has a stimulatory effect on bone formation. Zinc substituted hydroxyapatite is potentially a material, that can have the same effects. When zinc has been substituted into the hydroxyapatite and tricalcium phosphate (TCP) crystal lattices it has been found to inhibit osteoclasts in vitro, and to promote bone growth in vivo.

Despite the beneficial results, the clinical application of ion substituted ceramics and cements is limited due to low mechanical strength. Via coating of implants with ion substituted hydroxyapatite the higher mechanical strength of for example a metal can be combined with the properties of the ion substituted hydroxyapatite.

Therefore, ionic substituted hydroxyapatite as coating on implants is of interest to pursue. Such coatings produced so far were prepared using plasma spraying [9], sol-gel [10], magnetron cosputtering [11], pulsed-laser deposition [12], and micro-arc oxidation techniques [13]. These coating techniques do have some drawbacks. For example, the coatings are relatively thick and brittle, and also possess chemical defects. They do not always adhere well to the substrates. Furthermore, such coatings could not be evenly and uniformly applied to surfaces with complex geometries, such as porous and undercut surfaces. Furthermore, high temperature treatment is essential to produce these coatings, which puts constraints on the substrate materials that can be used. To overcome some of these drawbacks, low temperature processes have been applied to produce hydroxyapatite ceramic coatings through solution derived method. Bunker et al have discovered a technique for preparation of octacalcium phosphate coating by incubating the substrate in a solution containing calcium chloride [14]. Other examples are disclosed in U.S. Pat. No. 6,905,723 B2 [15] and No. 6,569,489 B1 [16] in which hydroxyapatite coatings are produced using a solution containing Ca²⁺, PO₄³⁻, Sr, Na⁺, K⁺, HCO₃₋, Cl⁻ and Mg²⁺.

Biomineralization is a natural self-assembly process of biomineral formation in water based solution. In the human body all normal and most pathological calcifications consist of calcium phosphate compounds. However, the hydroxyapatite content of bone is not stoichiometric calcium phosphate, but instead a calcium deficient and multi-substituted hydroxyapatite which is formed through biomineralization.

U.S. Pat. No. 6,569,481 and WO 9741273 describe methods to coat biomedical implants with hydroxyapatite (hydroxyapatite) coatings via a biomineralization process. The resulting coatings can optionally also contain silicate or sulphate. The description in said documents does not describe how the ion solutions are made, what source is used to create the ion solutions in order to form the hydroxyapatite coatings and no specific conditions of the implant surface to facilitate the coating deposition.

US 2004/0241314 (U.S. Pat. No. '314) shows a method for providing a bioactive implant with a strontium substituted apatite coating. The method involves incubating a surface not coated with a calcium containing compound into a composition comprising strontium, calcium and phosphate ions and a liquid carrier. The composition may further contain a biological agent, sodium, magnesium, carbonate, hydroxyl, chloride, fluoride ions or mixtures thereof.

### SUMMARY OF THE INVENTION

The present invention relates to a method of coating an implant with an ion substituted calcium phosphate with a controlled morphology.

A first aspect of the present invention is a method for forming a surface coating of an ion substituted calcium phosphate with controlled morphology on a substrate comprising the steps:
a. providing said substrate;
b. pre-treating said substrate in order to create an activated surface;
c. providing an aqueous solution comprising calcium ions, magnesium ions, phosphate ions and one or more of substitution ions selected from Sr²⁺, Si⁴⁺, F⁻, Ba²⁺, Fe³ and Zn²⁺, and optionally further comprising one or more of ions selected from sodium ions, potassium ions, chloride ions, carbonate ions and sulfate ions, having an initial pH in the range of 2.0 to 10.0 and a temperature of 20° C. to 100° C.; and
d. incubating at least a portion of the substrate in the aqueous solution for a period of time sufficient for the coating to be formed.

In one embodiment of the present invention the method for forming a surface coating of an ion substituted calcium phosphate with controlled morphology on a substrate comprises the steps:
a. providing a substrate;
b. pre-treating said substrate in order to create an activated surface;
c. providing an aqueous solution comprising calcium ions, magnesium ions, phosphate ions and one or more of substitution ions selected from Sr²⁺, Si⁴⁺, F⁻, Ba²⁺, Fe³ and Zn²⁺, and optionally further comprising one or more of ions selected from sodium ions, potassium ions, chloride ions, carbonate ions and sulfate ions, having an initial pH in the range of 6.0 to 8.0 and a temperature of 20° C. to 100° C.;
d. incubating at least a portion of the substrate in the first aqueous solution for a period of time sufficient for a first coating to be formed;
e. providing a second aqueous solution comprising calcium ions, magnesium ions, phosphate ions and one or more of substitution ions selected from Sr²⁺, Si⁴⁺, F⁻, Ba²⁺, Fe³ and Zn²⁺, and optionally further comprising one or more of ions selected from sodium ions, potassium ions, chloride ions, carbonate ions and sulfate ions, wherein the solution has an initial pH in the range of 6.0 to 8.0 and a temperature of 20° C. to 100° C.; and
f. incubating at least a portion of the substrate in the second aqueous solution for a period of time sufficient for a second layer of coating to be formed.

In another embodiment of the present invention the method steps c) to f) are repeated in order to create additional layers optionally containing another coating chemistry and morphology.

In another embodiment of the present invention the pre-treating involves formation of a calcium phosphate layer.

In another embodiment of the present invention the pre-treatment involves heat treatment, hydrolysis, oxidation, acid or base treatment, anodic oxidation, UV radiation, CVD, sol-gel or PVD.

In yet another embodiment of the present invention the substrate has charged groups on the surface.

In another embodiment of the present invention the charged groups are a result of a pre-treatment of the substrate surface.

In another embodiment of the present invention the immersion time in each solution is up to 2 weeks, preferably less then 1 week and more preferable less than 3 days.

In another embodiment of the present invention the concentration of:

calcium ions is in the range 0.01-25 10⁻³M, preferably 0.5-2.5 10⁻³M;
magnesium ions is in the range 0.01-15 10⁻³M, preferably 0.2-1.5 10⁻³M;
sodium ions is in the range 0.01-1420 10⁻³M, preferably 100-150 10⁻³M;
potassium ions is in the range 0.01-1420 10⁻³M, preferably 1.0-5.0 10⁻³M;
chloride ions is in the range 0.01-1030 10⁻³M, preferably 100-150 10⁻³M;
phosphate ions is in the range 0.01-10 10⁻³M, preferably 1.0-10 10⁻³M;
carbonate ions is in the range 0.01-270 10⁻³M, preferably 1.0-50 10⁻³M;
sulfate ions is in the range 0.01-5 10⁻³M, preferably 0.1-1.0 10⁻³M.

Another aspect of the present invention is an ion substituted coating comprising calcium, magnesium, phosphate and one or more of strontium, silicon, fluoride, barium, iron and zinc and optionally one or more of sodium, potassium, chloride, carbonate and sulfate.

In another embodiment the cationic substitution of calcium is up to 80%, preferably 25-60%.

In another embodiment the anionic substitution of phosphate and hydroxide is up to 30%, preferably 10-25%.

In one embodiment the coating contains 0-5%, preferably 1.5-3% of fluoride, or 0-10%, preferably 3-8% strontium, or 0-5%, preferably 0.5-2% silicon, or combinations thereof.

In another embodiment the morphology of the coating is in the form of sheets, flakes, spheres, porous structures, spikes or rods or a combination thereof.

In another embodiment the coating comprises multiple layers.

In another embodiment the coating is bioresorbable.

Another aspect of the present invention is the use of the ion substituted calcium phosphate coating as a drug and/or ion delivery system.

### BRIEF DESCRIPTION OF THE FIGURES

FIG. 1. XRD patterns of heat treated titanium plates incubating into the 0.06 mmol/l and 0.6 mmol/l Sr PBS solution at 37° C. (A), and 60° C. (B), respectively, for 1 week.

(*: specific peak of calcium phosphate.)

FIG. 2. XRD patterns of heat treated titanium plates incubating into the 0.06 mmol/l and 0.6 mmol/l Sr PBS solution at 37° C. (A), and 60° C. (B), respectively, for 2 weeks.

(*: specific peak of calcium phosphate.)

FIG. 3. SEM images of heat treated titanium surface, magnification (A) 3,000 X, (B) 10,000 X.

FIG. 4. SEM images of heat treated titanium surface after incubating into 0.06 mM strontium PBS for 1 week at 37° C., magnification 10,000 X.

FIG. 5. SEM images of heat treated titanium surface after incubating into 0.06 mM strontium PBS for 2 weeks at 37° C., magnification 10,000 X.

FIG. 6. SEM images of heat treated titanium surface after incubating into 0.06 mM strontium PBS for 1 week at 60° C., magnification (A) 1,000 X, (B) 30,000 X.

FIG. 7. SEM images of heat treated titanium surface after incubating into 0.06 mM strontium PBS for 2 weeks at 60° C., magnification (A) 1,000 X, (B) 30,000 X.

FIG. 8. SEM images of heat treated titanium surface after incubating into 0.6 mM strontium PBS for 1 week at 37° C., magnification (A) 10,000 X, (B) 45,000 X.

FIG. 9. SEM images of heat treated titanium surface after incubating into 0.6 mM strontium PBS for 1 week at 60° C., magnification (A) 10,000 X, (B) 50,000 X.

FIG. 10. XRD patterns of PVD treated titanium plates incubating into the 0.06 mmol/l and 0.6 mmol/l Sr PBS solution at 37° C. (A), and (B) 60° C., respectively, for 1 week.

(*: specific peak of calcium phosphate.).

FIG. 11. XRD patterns of PVD treated titanium plates incubating into the 0.06 mmol/l and 0.6 mmol/l Sr PBS solution at 37° C. and 60° C., respectively, for 2 weeks.

(*: specific peak of calcium phosphate.).

FIG. 12. SEM images of PVD treated titanium surface, magnification (A) 1,000 X, (B) 30,000 X.

FIG. 13. SEM images of PVD treated titanium surface after incubating into 0.06 mM strontium PBS for 1 week at 60° C., magnification (A) 3,000 X, (B) 30,000 X.

FIG. 14. SEM images of PVD treated titanium surface after incubating into 0.6 mM strontium PBS for 1 week at 60° C., magnification (A) 3,000 X, (B) 30,000 X.

FIG. 15. SEM images of PVD treated titanium surface after incubating into 0.06 mM strontium PBS for 2 weeks at 60° C., magnification (A) 1,000 X, (B) 30,000 X.

FIG. 16. SEM images of PVD treated titanium surface after incubating into 0.6 mM strontium PBS for 2 weeks at 60° C., magnification (A) 1,000 X, (B) 30,000 X.

FIG. 17. SEM images of PVD treated titanium surface after incubating into silicon PBS.

FIG. 18. SEM image of heat treated titanium surface after incubating into Si and Sr PBS for 1 week at 37° C.

FIG. 19. Illustrates Si and Sr ion signals on the biomineralized surface from TOF-SIMS.

FIG. 20. SEM images of fluorapatite growth on heat-treated titanium surfaces. Heat-treated titanium surface (A). Ti plates soaked into phosphate buffered solution containing 0.2 mM F⁻ at 60° C. for 12 hours (B), 1 day (C), 1 week (D).

FIG. 21. Ion-release curves from strontium ion substituted calcium phosphate coatings.

FIG. 22. Ion-release curves from silicon ion substituted calcium phosphate coatings.

FIG. 23. Ion-release curves from fluoride ion substituted calcium phosphate coatings.

FIG. 24. SEM showing surface morphology of the TiO₂/Ti substrates after soaking in PBS containing 0.6 mM Sr ions; (A) 12 hours, first layer (thin and dense); (B) 2 weeks, second layer (porous).

FIG. 25. XRD patterns of oxidized substrates soaking into the 0.6 and 0.06 mmol/l Sr-PBS solution at 37° C. and 60° C., respectively, for 1 week and 2 weeks. (A: apatite, T: titanium).

FIG. 26. XPS spectra for strontium substituted apatite/titanium dioxide coating on titanium plates (thermal oxidation, 0.6 mM Sr in PBS, 60° C. for 1 week).

### DETAILED DESCRIPTION OF THE INVENTION

In the present application the term “biomineralization” refers to the formation of a mineral substance through self-assembly. In the present application biomineralization does not necessarily have to involve living organisms and can be performed both in vitro and in vivo.

In the present application the term “calcium phosphate” refers to mineral substances containing calcium and phosphate and includes hydroxyapatite or brushite or monetite or amorphous calcium phosphate coatings or combinations thereof.

In the present application the wording “ion substitution” refers to the process wherein an ion within a substance is exchanged for another ion with the same (i.e. positive or negative) charge.

The present invention is based on the understanding that a biomineralized layer will have beneficial effects on the anchoring of an implant to a host tissue and bone regeneration. Biomineralization combined with ion substitution will have advantages due to ion substituted calcium phosphate coatings having a high similarity to the natural mineral of bone which is obtained through a biomineralization process. Furthermore, because the biomineralization process takes place in an aqueous solution according to the present invention, it is applicable to any open surface and is not limited by any complex geometry of the implant. The method according to the present invention is also a low temperature technique, which is energy efficient and applicable to temperature sensitive substrate materials. The present invention specifically relates to the combination of coating chemistry and morphology obtained via applying the method and materials as described in the invention.

Unlike U.S. Pat. No. '314 mentioned above, the present invention provides a strategy to control the morphology of the coating. The morphology, as will be discussed further down, is an important factor when it comes to tissue response in vivo and the control of the morphology facilitates the use of the coating as a drug and/or ion delivery system. The present invention, unlike U.S. Pat. No. '314, discloses a pre-treatment step in combination with ion substitution. This is a novel technique and provides a method for controlled morphology coating.

The aim of the pre-treatment is to activate the surface, i.e. to achieve optimal growth conditions for the calcium phosphate coating. Preferably the surface should have a negative surface charge in the incubating solution. For Ti-implants for example this involves heat treatment, hydrolysis, anodic oxidation, acid or base treatment, UV radiation, or CVD, sol-gel deposition or PVD with the main aim of forming a crystalline titanium dioxide coating with small grain size (see for example WO 2005/055860 and U.S. Pat. No. 6,183,255, J Biomed Mater Res 82A: 965-974 (2007), Applied Surface Science Vol 255 Issue 17 (2009) Pages 7723-772). The same or similar treatments could also be applied for other substrate materials and the pre-treatment could involve more than one treatment. Pre-treatment of surfaces in aqueous solution, optionally comprising groups such as —OH, —COOH, —NH₂ is pursued as described in the art. The pre-treatment of the present invention causes a faster coating process and results in a more even, for example thickness, coating. It is believed, without being bound by any theory, that the pre-treatment creates nucleation points wherein the coating can starts. A non pre-treated surface may be more dependent on local variations on the substrate surface, said variations could be a result of handling.

Additionally, the pre-treatment may comprise forming a calcium phosphate layer. This layer can be formed according to any technique known to a person skilled in the art. For example, the calcium phosphate layer may be formed by incubating the substrate in a solution containing only calcium ions, magnesium ions and phosphate ions at a pH between 2-10 and at a temperature between 20-100° C. Said calcium phosphate layer is preferably formed on a surface containing charges.

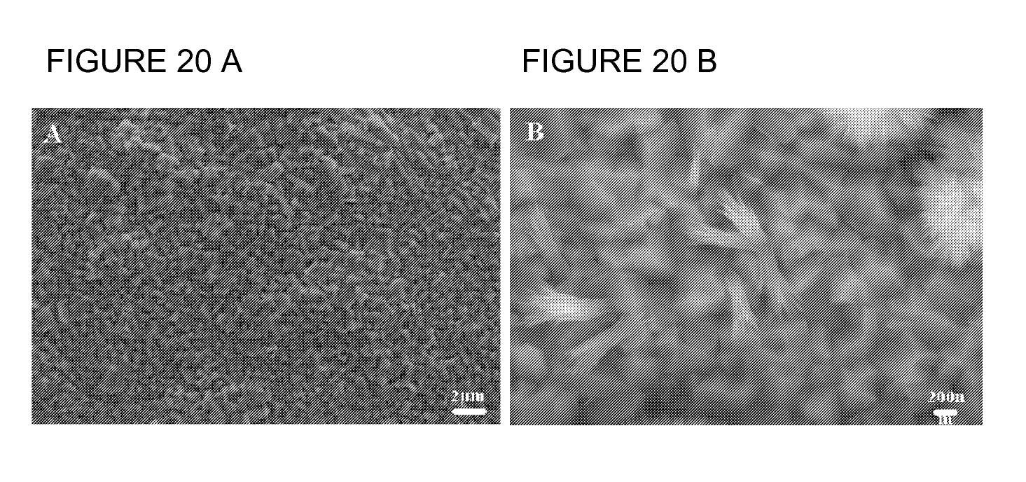

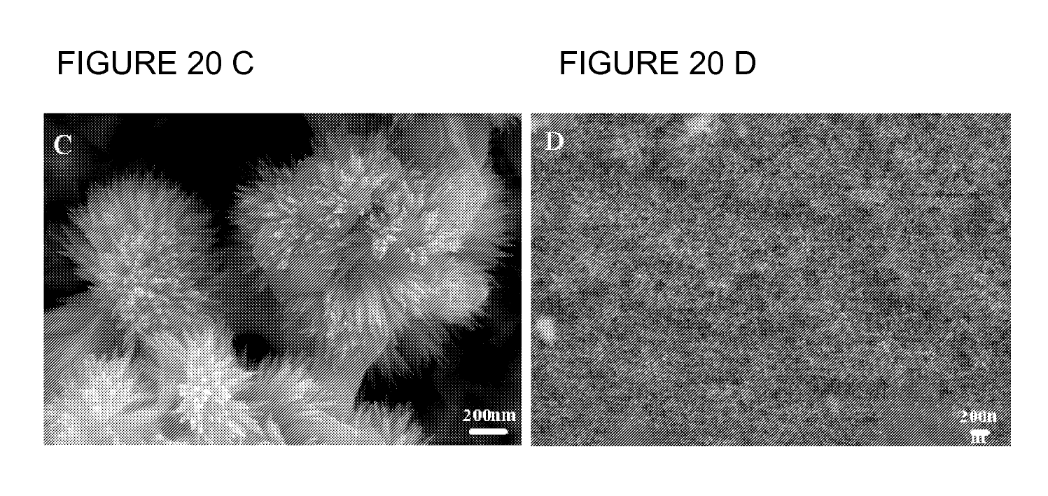

This invention provides a new technique for preparing an ion substituted calcium phosphate using substitution ions such as F⁻, Sr²⁺, Si⁴⁺, Zn²⁺, Ba²⁺, Fe³+, Mg²⁺, Cl⁻ and CO₃²⁻ on implants. A person skilled in the art knows what substitution ions that can be used. According to the present invention the morphology depends on the ion or ions used in the ion substitution step. For example when substituting with F-ions the morphology could be described as having spikes or rods, FIG. 20, while silicon generates a morphology that could be described as sheets or flakes,

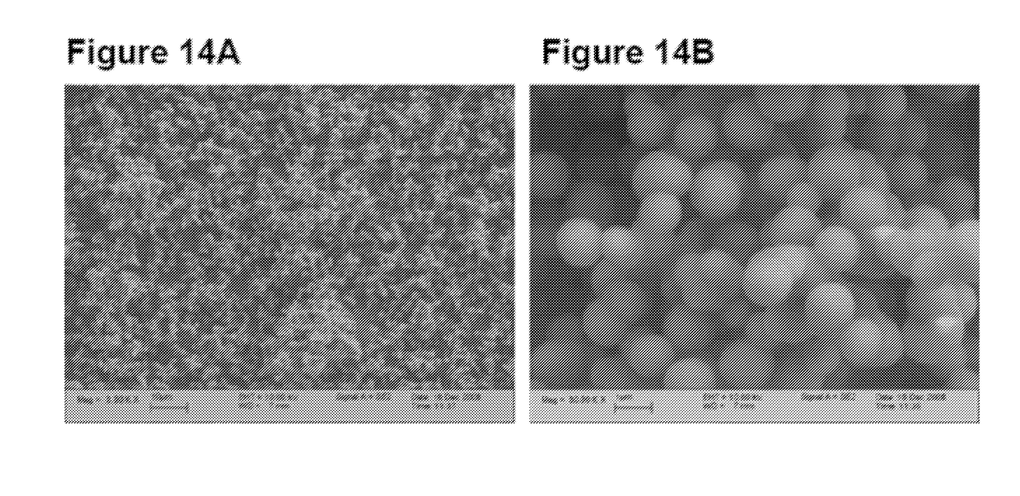

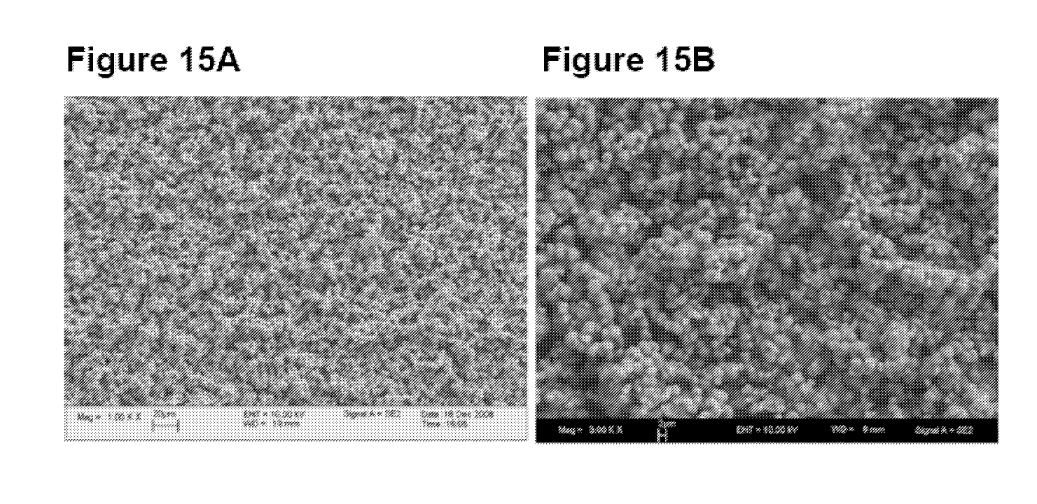

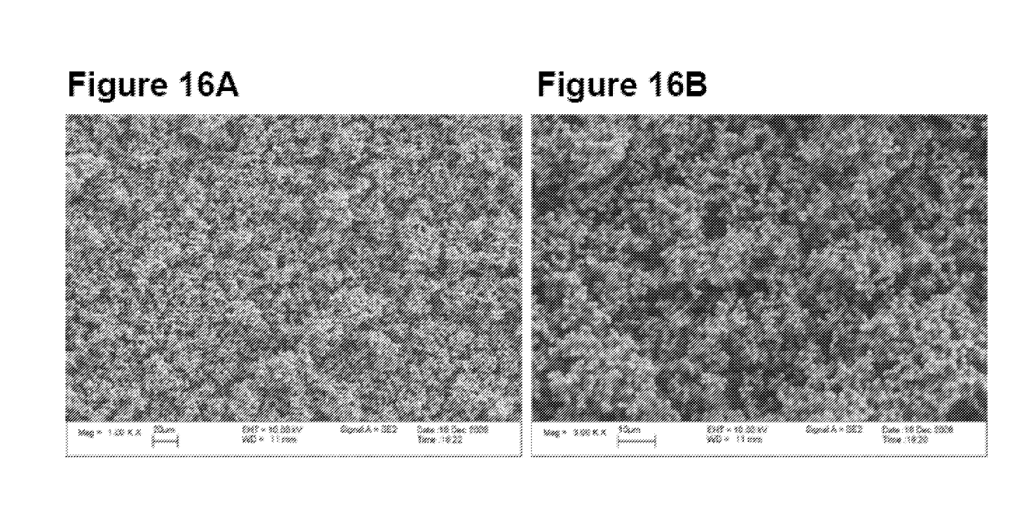

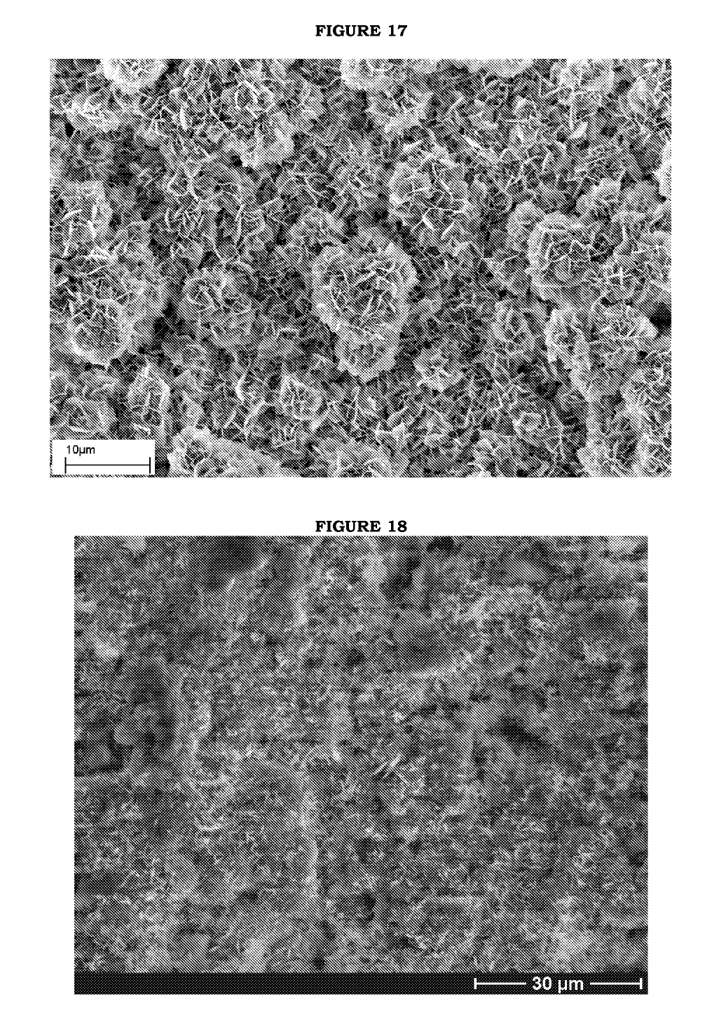

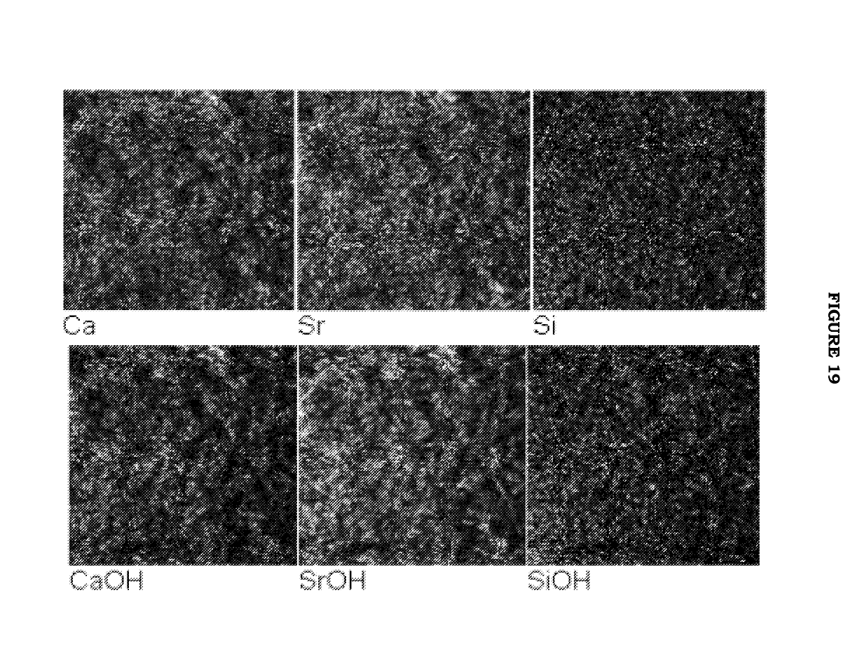

FIG. 17, and strontium results in a structure with spherical particles and pores, see for example FIGS. 14 and 15. Additionally, different ions result in different solubility constants for the coating. It has previously been shown that for example a fluoride substitution results in a lowering of the solubility constant while addition of strontium or silicon will increase the solubility constant in comparison with hydroxyapatite.

The pore size could also be controlled by varying the substituting ions, temperature and immersion time.

This new technique is based on a biomineralization process using a modified simulated body fluid and phosphate buffer solution containing the cationic and anionic substituted ions in the calcium phosphate coating. The cationic substitution ions are Sr²⁺, Si⁴⁺, Zn²⁺, Ba²⁺, Fe³⁺ or Mg²⁺; and the anionic substitution ions are F⁻or CO₃²⁻. The source for the ion substitutions can be soluble salts and slight-soluble salts containing the ions to be substituted, such as SrCl₂, SrCO₃, Sr(NO₃)₂, Na₂SiO₃, calcium silicate (CaOSiO₂, CaO(SiO₂)₂, CaO(SiO₂)₃, ZnCl₂, ZnSO₄, BaCl₂, FeCl₃, Fe(NO₃)₃, Na CO₃, NaF, Na₂FPO₄.

The formation of ionic substituted calcium phosphate coatings through a biomineralizing method includes incubating for example a bioactive implant specimen in a mineralizing solution, such as modified simulated body fluid (SBF) and/or phosphate buffer solution (PBS) (Table 1), containing different cations and/or anions. The mineralizing solution may include the major inorganic ions present in human body, namely Na⁺, K⁺, Ca²⁺, HCO₃⁻, HPO₄²⁻, SO₄²⁺.

TABLE 1
Ion concentrations in blood plasma, modified
SBF, and modified PBS (10⁻³ M)

| | Blood plasma | | |
|---|---|---|---|
| Ion | (mmol/l) | SBF (mmol/l) | PBS* (mmol/l) |
| Na⁺ | 142 | 142 | 145 |
| K⁺ | 5 | 5 | 4.2 |
| Mg²⁺ | 1.5 | 1.5 | 0.49 |
| Ca²⁺ | 2.5 | 2.5 | 0.91 |
| Cl⁻ | 103 | 148 | 143 |
| HCO³⁻ | 27 | 4.2 | |
| HPO₄²⁻ | 1 | 1 | 9.6 |
| SO₄²⁻ | 0.5 | 0.5 | |

*purchased from Sigma-Aldrich.

As described above, the present invention provides a method for forming a surface coating of an ion substituted calcium phosphate with controlled morphology on a substrate comprising the steps:
a) providing said substrate;
b) pre-treating said substrate in order to create an activated surface;
c) providing an aqueous solution comprising calcium ions, magnesium ions, phosphate ions and one or more of substitution ions selected from Sr²⁺, Si⁴⁺, F⁻, Ba²⁺, Fe³ and Zn²⁺, and optionally further comprising one or more of ions selected from sodium ions, potassium ions, chloride ions, carbonate ions and sulfate ions, having an initial pH in the range of 2.0 to 10.0 and a temperature of 20° C. to 100° C.; and
d) incubating at least a portion of the substrate in the aqueous solution for a period of time sufficient for the coating to be formed.

The biomineralization process can be divided into multiple steps wherein each step may contain solutions with different ions and ion concentrations. The procedure comprises incubating the substrate in the mineralizing solution for example for 1-7 days preferably 1-3 days, and transferring it into the aqueous solution containing the substitution ions for example for 1-7 days preferably 1-3 days. This procedure is repeated until the thickness and/or the ion content in the new coating has reached a target value.

As described above, the present invention may also be performed in several steps in order to form a coating, with controlled morphology, of an ion substituted calcium phosphate on a substrate comprising the steps;
a) providing a substrate;
b) pre-treating said substrate in order to create an activated surface;
c) providing an aqueous solution comprising calcium ions, magnesium ions, phosphate ions and one or more of substitution ions selected from Sr²⁺, Si⁴⁺, F⁻, Ba²⁺, Fe³ and Zn²⁺, and optionally further comprising one or more of ions selected from sodium ions, potassium ions, chloride ions, carbonate ions and sulfate ions, having an initial pH in the range of 6.0 to 8.0 and a temperature of 20° C. to 100° C.;
d) incubating at least a portion of the substrate in the first aqueous solution for a period of time sufficient for a first coating to be formed;
e) providing a second aqueous solution comprising calcium ions, magnesium ions, phosphate ions and one or more of substitution ions selected from Sr²⁺, Si⁴⁺, F⁻, Ba²⁺, Fe³ and Zn²⁺, and optionally further comprising one or more of ions selected from sodium ions, potassium ions, chloride ions, carbonate ions and sulfate ions, wherein the solution has an initial pH in the range of 6.0 to 8.0 and a temperature of 20° C. to 100° C.; and
f) incubating at least a portion of the substrate in the second aqueous solution for a period of time sufficient for a second layer of coating to be formed. Optionally, steps c) to f) can be repeated any number of times in order to create additional layers containing another coating chemistry and morphology.

A preferred strategy when forming a coating on a surface according to the present invention is to activate the surface prior to the incubating. The activation could involve creating charge groups, negative or positive groups. Preferably, the surface should be negatively charged when soaking below approximately 40° C. and positively charged at higher temperatures. This activation will enhance the surface ion attraction and results in an even coating.

Prior to step b) the surface of the substrate may be cleaned in a manner best suitable for the substrate material to achieve an optimal surface for coating growth and adhesion, for example formation of a crystalline titanium dioxide coating on metal implants. Additionally, after each step c) and f) the surface may be cleaned and then rinsed, or just rinsed, with for example de-ionized water, or any other suitable solvent, and dried.

As mentioned above, the concentration of:

calcium ions is in the range 0.01-25 10⁻³M, preferably 0.5-2.5 10⁻³M;
magnesium ions is in the range 0.01-15 10⁻³M, preferably 0.2-1.5 10⁻³M;
sodium ions is in the range 0.01-1420 10⁻³M, preferably 100-150 10⁻³M;
potassium ions is in the range 0.01-1420 10⁻³M, preferably 1.0-5.0 10⁻³M;
chloride ions is in the range 0.01-1030 10⁻³M, preferably 100-150 10⁻³M;
phosphate ions is in the range 0.01-10 10⁻³M, preferably 1.0-10 10⁻³M;
carbonate ions is in the range 0.01-270 10⁻³M, preferably 1.0-50 10⁻³M;
sulfate ions is in the range 0.01-5 10⁻³M, preferably 0.1-1.0 10⁻³M.

The concentration of substituting cations, i.e. Sr²⁺, Si⁴⁺, Zn²⁺, Ba²⁺, Fe³, is in the range of 0.01-0.1 10⁻³M, and the concentration of substituting anions, i.e. F⁻ is in the range of 1-100 10⁻³M.

The amount of cations and/or anions in the substitution solution may be adapted depending on the wanted substitution content. The cationic substitution of calcium could be up to 80%, and the anionic substitution of phosphate and hydroxide could be up to 30%.

The method according to the invention can be applied to a variety of substrates, including titanium, titanium alloys, other metal and alloys, bioceramics, bioactive glasses, and polymers.

The method according to the invention can preferably be applied to bone anchoring implants, where an enhanced and permanent bone healing is desired to obtain a good clinical function. Examples of such applications are dental implants, craniofacial implants, or orthopedic implants.

The thickness of the ionic substituted calcium phosphate coating prepared according to the present invention can be controlled in the range 10 nm to 100 μm by immersion time, temperature and ion concentrations of the process solution. Increasing the immersion time, temperature and ion concentration will result in an increase in coating thickness. When the thickness of the coating becomes too thick, the mechanical properties of the coating decrease and the coating becomes more brittle. Therefore the immersion time should be optimized in order to get the right thickness and mechanical properties. A preferred thickness, in respect of mechanical strength and adherence, of the coating is below 10 μm, more preferably below 5 μm.

The method according to the present invention is performed at a temperature from 20° C. to 100° C., preferably from 37° C. to 60° C. The immersion time, i.e. the time the substrate is in the biomineralization and ion substitution solution is from 1 day to 2 weeks, preferably 1 day to 7 days and more preferably 1 to 3 days. The method according to the invention can be applied to coat surfaces with complex geometries, such as porous materials and undercuts. The present invention facilitates a uniform thickness of the coating independently of the geometry of the substrate surface.

The method according to the invention is not only applicable for producing single ionic substituted calcium phosphate coatings but also two, three, and four ionic substituted calcium phosphate coatings. Additionally, the method allows coating of only a part of the implant, as well as coating different parts of the implant with coatings with different ion substitutions and/or thicknesses. This makes it possible to tailor the properties of the implant even further since various parts of the implant might be in contact with different tissues. Therefore the chemistry, morphology and mechanical properties of different parts of the coating can be adapted in order to optimize the function of the implant.

The present invention further provides a crystalline ion substituted calcium phosphate surface coating produced by the method according to the invention wherein the coating comprising calcium, magnesium, phosphate and one or more of strontium, silicon, fluoride, barium, iron and zinc and optionally sodium, potassium, chloride, carbonate and/or sulfate. Preferably, the coating further contains 0-5%, preferably 1.5-3% of fluoride, or 0-10%, preferably 3-8% strontium, or 0-5%, preferably 0.5-2% silicon, or combinations thereof.

The present invention further provides a crystalline ion substituted calcium phosphate surface coating with specific characteristics determined by,
a) X-ray diffraction (XRD),
b) Scanning electron microscope (SEM);
c) X-ray Photoelectron Spectroscopy (XPS); and/or
d) Time-of-Flight Secondary Ion Mass Spectroscopy (ToF-SIMS);

### Applications

Based on the wide range of surface coatings that can be produced with the invention several applications can be envisaged.

### As a Mechanical Enhancer for Implants

The use of the bioactive coatings with their beneficial biological effects makes them suitable to apply to biomedical implants. This includes temporary and permanent materials where the said coating can improve the bonding of the implant to tissues.

A special circumstance is the clinical need of a rapid and permanent bone healing around the implant and a rapid implant anchoring, e.g. dental, craniofacial and orthopedic implants. Examples of the latter include applications with spinal implants, arthroplasties, osteosynthesis applications and fixation devices, cartilage and subchondral bone defects, bone void fillers and other situations where an implant should fixate bony parts, augment bone and replace defects and allow functional loads to be applied. Of particular interest are implants in bone tissues that are compromised in some way, due to disease (e.g. osteoporosis, diabetes), trauma, aging and sequelae after treatment (e.g. radiotherapy).

Further, plausible situations are when the prognosis for successful implant treatment is smaller with less optimal implant surfaces. Also those situations where the anatomy of the patient results in a decreased success prognosis, such as in areas with small amounts of bone that can provide initial implant stability, the use of the invention will be advantageous.

The possibility offered by the invention to only coat parts of an implant, or to produce different types of coatings on different parts of an implant, opens up the possibility to tailor-make the surface properties of implants for optimal biological performance in respect of specific types of tissue and for individual patients. For a bone anchoring implant penetrating skin or mucosa the invention can be used to apply a coating exclusively to those parts of the implant that are in contact with bone. The use of different coatings on different parts of the implant can also be used for producing coatings that provide the optimal response depending on which type of bone tissue that different parts of the implant are in contact with. For a bone anchoring implant contacting both cortical bone and bone marrow, different parts of the implant can be supplied with different coatings designed to optimize the performance in these types of tissues.

### As a Drug and Ion Delivery System

Thanks to the controllable morphology of the coating, the coating itself can also serve as a deposit for delivering both drugs and ions in a controllable fashion.

The ion substituted coatings can when placed in vivo provide necessary ions to the surrounding tissue. Thus the coating function like a deposit for essential ions for bone formation, this can be tailored for specific control of the bone formation. These ions could be Ca, F, Zn, phosphate, chlorine, sulfate, Ba, Fe, K, Mg, Na, carbonate, strontium or silicon. The provision of said ions may enhance the bone regeneration, strengthening of bone, control the chemical stability of bone and possibly provide the implant with better anchoring to surrounding bone.

Additionally, a porous structure for example could be loaded with drugs. These drugs would then diffuse continuously or discontinuously depending on the morphology. The calcium phosphate coating could also be bioresorbable and therefore would allow a sustained and controllable release of a drug. Examples of drugs include bisphosphonates, statins, antibiotics, anti inflammatory, bone growth proteins and combinations thereof. The coating may be preloaded or loaded in the operating theatre at the time of implant placement.

Multilayer structures of the coating allow a tailoring of the drug/ion release system. The various layers may vary in morphology, density, thickness, chemistry, and of course in ion/drug content.

### EXAMPLES

### Example 1

### Deposition of Strontium Substituted Calcium Phosphate Coating on Heat Treated Titanium Surfaces

10 mm×10 mm titanium plates were treated using heat treatment (at 800° C. for 2 hours) to get titanium dioxide surface. The treated plates were first cleaned ultrasonically in acetone, followed in ethanol, and finally rinsed with de-ionized water and dried at 37° C. Two kinds of mineralizing solutions were obtained from the modified phosphate buffer solution (PBS) (see table 2). The low concentration of Sr PBS was 0.06 mmol/l. The high one was 0.6 mmol/l. The initial pH of the low one was 7.20 and 7.21 at 37° C. and 60° C., respectively. The initial pH of the high one was 7.19 and 7.15 at 37° C. and 60° C., respectively. Every two specimens were soaked into 40 ml preheated solution in a sealed plastic bottle, which were then put into the oven at 37° C. and 60° C., respectively. The plates were incubated for different time periods from 1 week to 2 weeks. All specimens were then rinsed with de-ionized water and dried in air. The specimens were then analyzed using thin-film X-ray diffractometry (TF-XRD), field emission scanning electron microscopy (FESEM), X-ray photoelectron spectroscopy (XPS) spectra, and time-of-flight secondary ion mass spectroscopy (TOF-SIMS).

TABLE 2
Ion concentrations in blood plasma,
PBS, and strontium PBS (10⁻³ M)

| Ion (mmol/l) | Na⁺ | K⁺ | Mg²⁺ | Ca²⁺ | Cl⁻ | HPO₄²⁻ | Sr²⁺ |
|---|---|---|---|---|---|---|---|
| Blood plasma | 142.0 | 5.0 | 1.5 | 2.5 | 103.0 | 1.0 | |
| PBS | 145.0 | 4.2 | 0.49 | 0.91 | 143 | 9.6 | — |
| 06Sr-PBS | 145.0 | 4.2 | 0.49 | 0.91 | 143 | 9.6 | 0.6 |
| 006Sr-PBS | 145.0 | 4.2 | 0.49 | 0.91 | 143 | 9.6 | 0.06 |

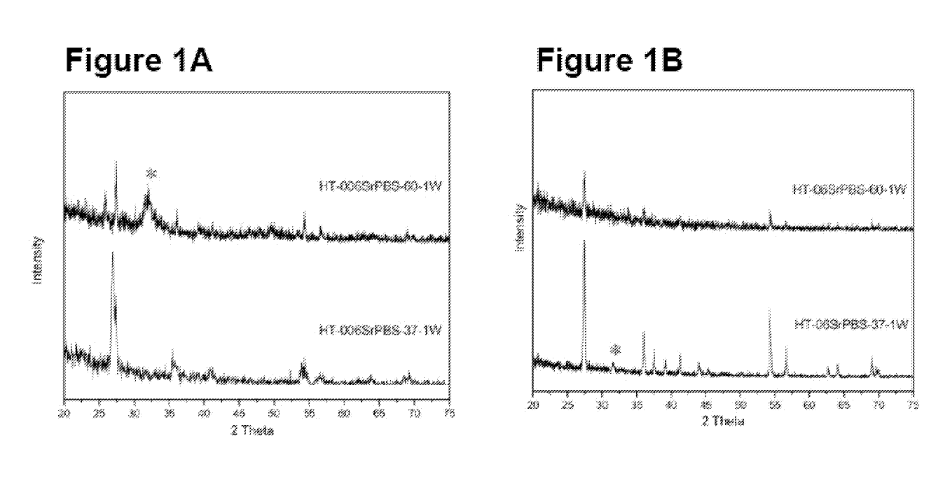

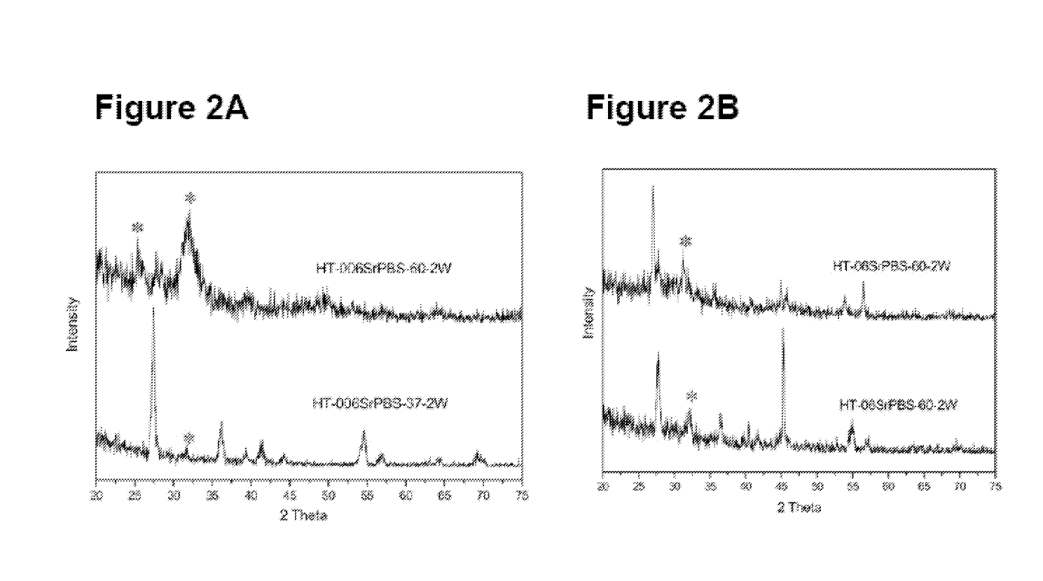

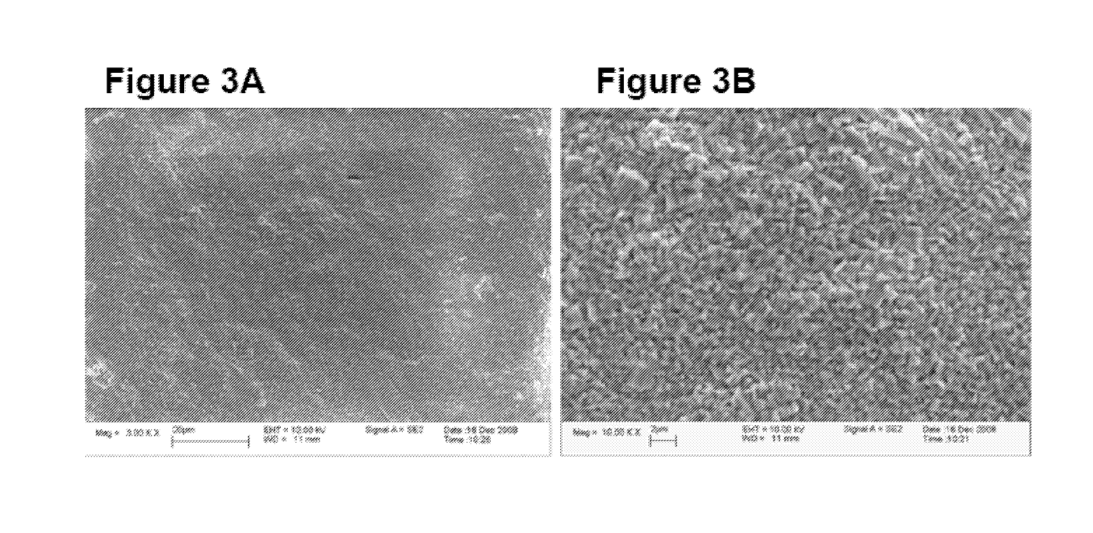

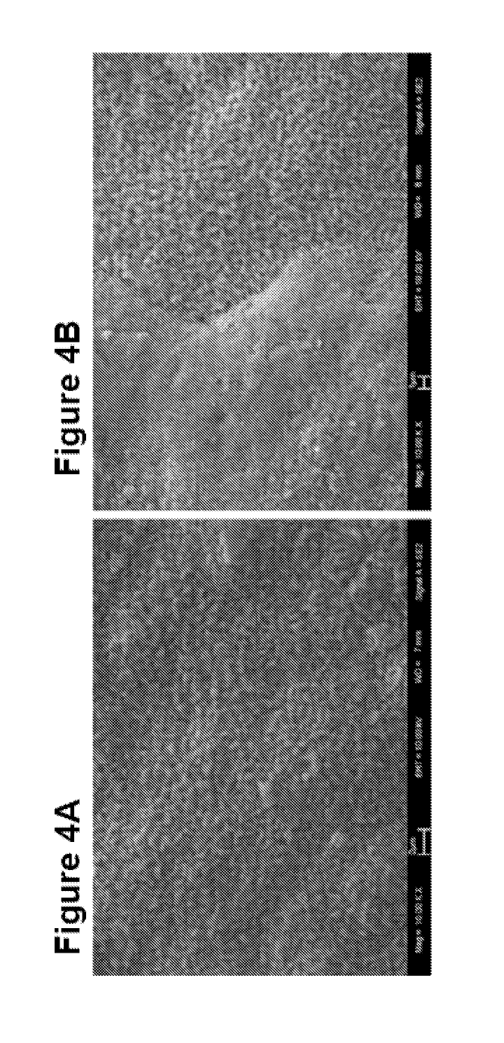

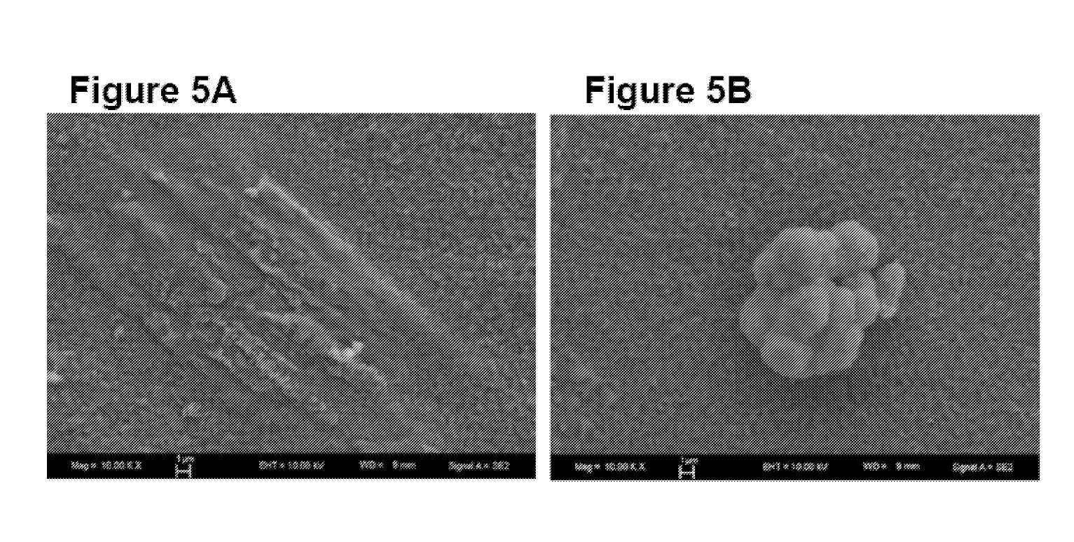

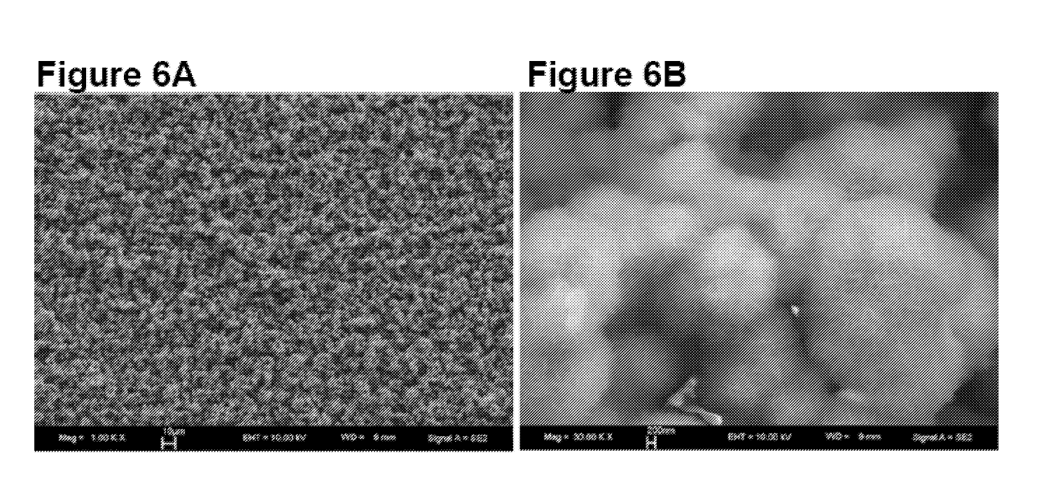

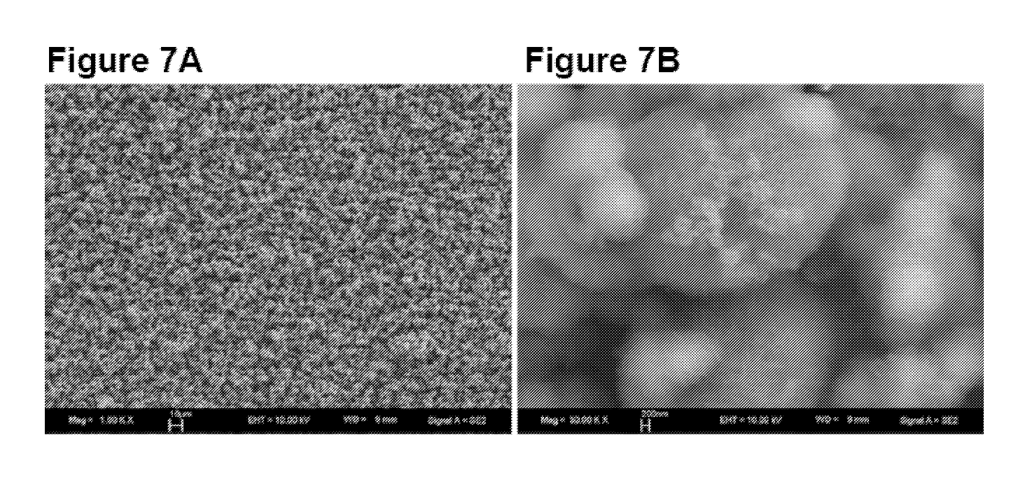

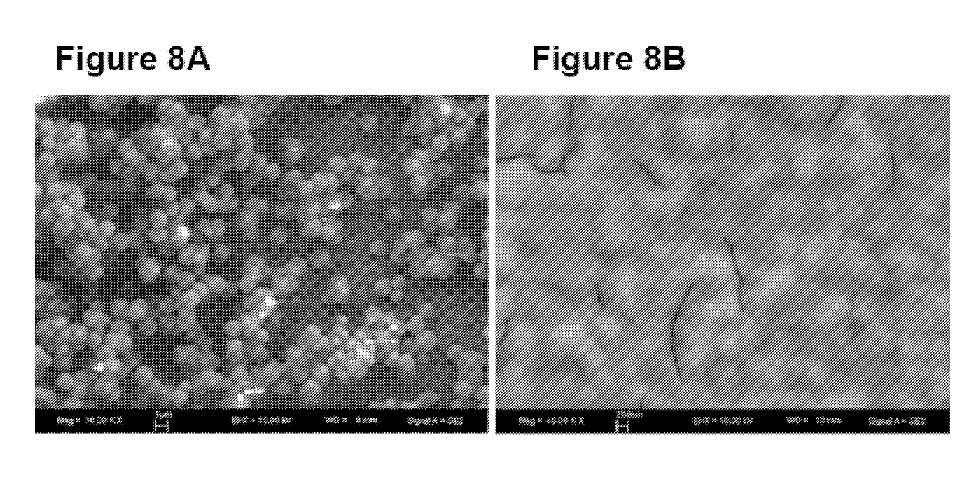

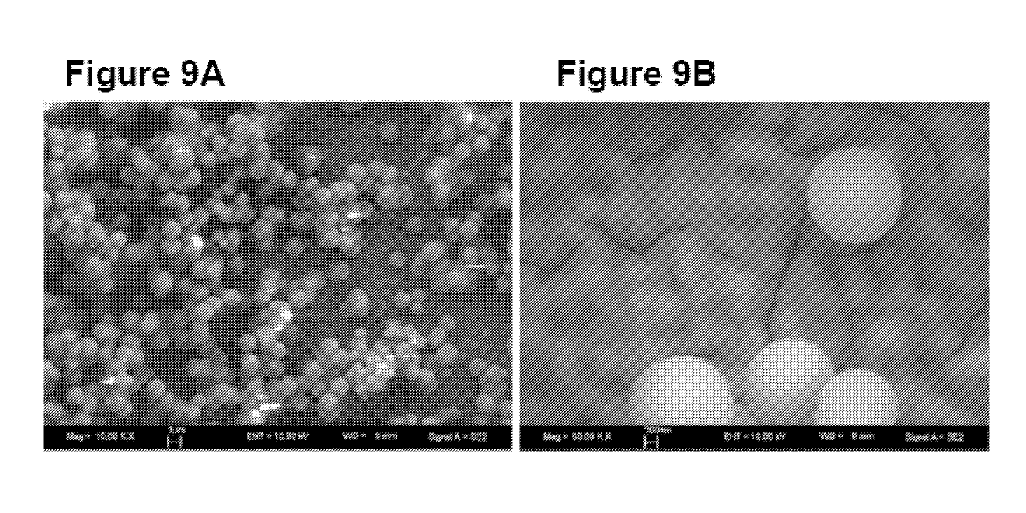

The results are shown in FIG. 1-9. The coating of biomineralized strontium substituted calcium phosphate was made up of two layers after soaked into the mineralizing solution for 1 and 2 weeks. The bottom layer was a thin and dense coating, and the upper layer was a loose and porous coating (FIGS. 8 and 9).

### Example 2

### Deposition of Strontium Substituted Calcium Phosphate Coating on PVD Treated Titanium Surfaces

The coating process was similar to example 1 but the substrates were PVD treated titanium plates.

The PVD treatment was as following:

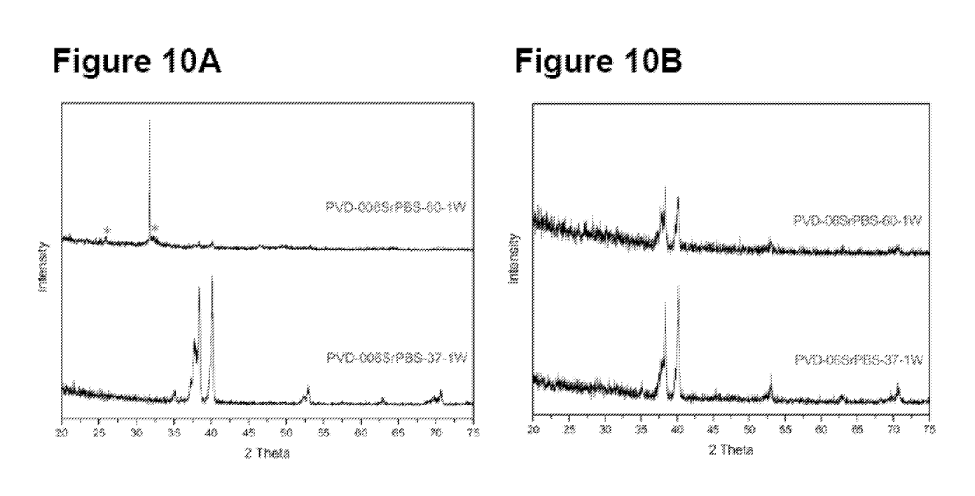

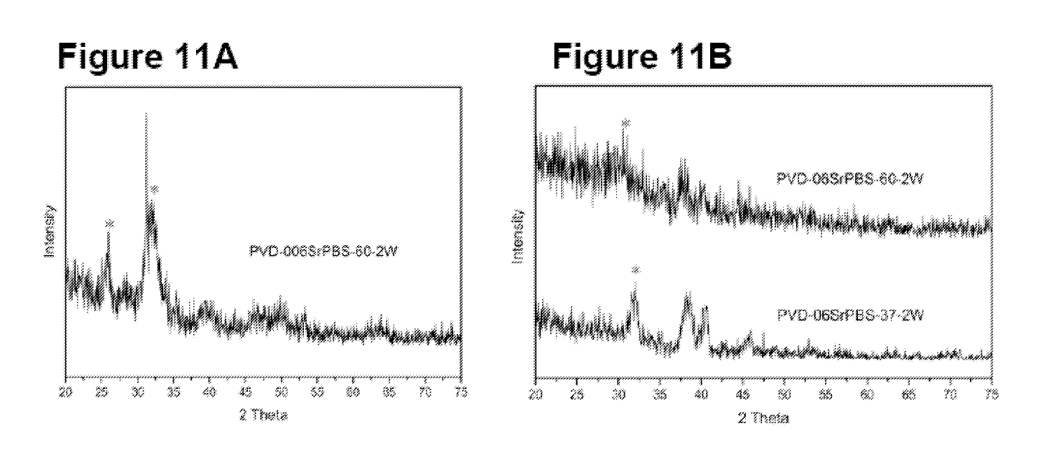

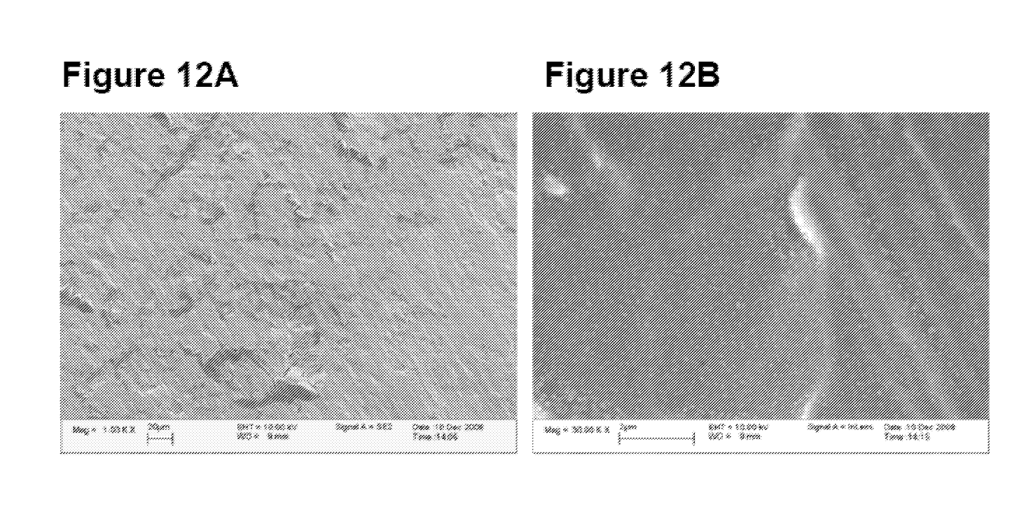

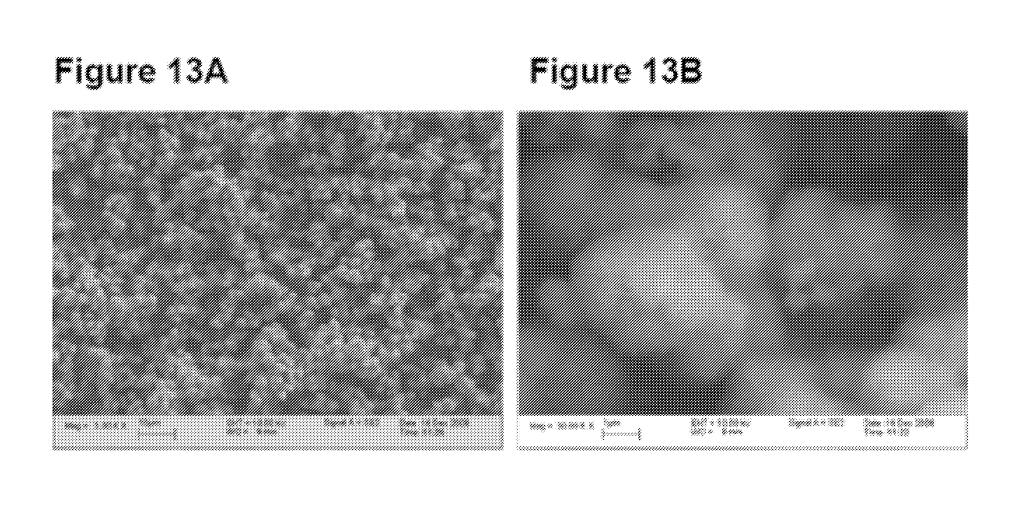

The titanium plates were placed in a PVD chamber (Baltzer 640R). The magnetron effect and the oxygen partial pressure during the coating step were 1.5 kW and 1.5×10⁻³ mbar, respectively. The setup of the PVD apparatus was optimised for maximum production of the rutile structure in the TiO₂ film. The results are shown in FIGS. 10-16.

### Example 3

### Deposition of Silicon Substituted Calcium Phosphate Coating on PVD Treated Titanium Surfaces

10 mm×10 mm titanium plates were treated using PVD treatment as described in Example 2 to get titanium dioxide surface. The treated plates were first cleaned ultrasonically in acetone, followed in ethanol, and finally rinsed with de-ionized water and dried at 37° C. The mineralizing solution containing silicate was obtained from the modified phosphate buffer solution (PBS). The modified PBS was prepared as the following:
(1) 3 mg tricalcium silicate was added into 40 ml PBS solution stirred overnight.
(2) The turbid solution was then centrifuged, and the supernatant solution was acted as the mineralizing medium.
(3) The pH value and composition of the solution were analyzed by pH meter and ICP-AES, respetively.

Every two specimens were soaked into 40 ml preheated solution in a sealed plastic bottle, which were then put into the oven at 60° C. for 1 week. All specimens were then rinsed with de-ionized water and dried at 37° C. The specimens were then analyzed using thin-film X-ray diffractometry (TF-XRD), field emission scanning electron microscopy (FESEM), X-ray photoelectron spectroscopy (XPS) spectra, and time-of-flight secondary ion mass spectroscopy (TOF-SIMS). The results are shown in FIG. 17.

### Example 4

### Preparation of Ion Substituted Calcium Phosphate Coatings

Other preferred ionic (Mg²⁺, Zn²⁺, Ba²⁺, Fe³⁺, CO₃²⁻, F⁻, Cl⁻, etc.) substituted calcium phosphate coatings were prepared by the method could be obtained by the similar process as example 1-3. The solution containing different ions could be prepared through dissolving soluble salts in modified SBF and PBS.

These ionic substituted calcium phosphate coatings are prepared on substrates such as bioactive ceramics (hydroxyapatite, tricalcium phosphate (TCP), calcium silicate, zirconia), bioactive glasses (45S5 bioglass@, AW glass-ceramics, bioactive 58S glass), metals (titanium, titanium alloys, stainless steel, CoCrMo alloy), carbon and polymers (collagen, glutin, PLGA, PGA).

### Example 5

### Preparation of Si and Sr Co-Substituted Calcium Phosphate Coatings

The substrate treatment process was performed as described in example 1.

The solution containing silicate and strontium was obtained from the modified phosphate buffer solution (PBS). The silicate source was sodium silicate solution, and the strontium source was strontium nitrate in this example. The Si ion concentration was controlled in 0.075-0.15 mM, and the Sr ion concentration was controlled in 0.06-0.6 mM.

The specimens were soaked into 40 ml preheated solution in a sealed plastic bottle, which were then put into the oven at 37° C. for 1 week. All specimens were rinsed with de-ionized water and dried at 37° C. after incubating. The specimens were then analyzed using thin-film X-ray diffractometry (TF-XRD), field emission scanning electron microscopy (FESEM), X-ray photoelectron spectroscopy (XPS) spectra, and time-of-flight secondary ion mass spectroscopy (TOF-SIMS).

The results are shown in FIGS. 18-19. The analysis showed that the co-substituted apatite coating was formed on the substrate, see the SEM images. The TOF-SIMS results showed that there were Si and Sr ion signals on the surface.

TheECa/ESr was around 0.83, and the ΣSiOx/ΣPOx was around 0.08. These results showed that the Si and Sr co-substituted apatite coating was formed on the heat-treated Ti substrates.

### Example 6

Bone mineral is a multi-substituted calcium phosphate. One of these ion substitutions, strontium, has been proven to increase bone strength and decrease bone resorption. Biomimetics is a potential way to prepare surfaces that provide a favorable bone tissue response, thus enhancing the fixation between bone and implants. Here we prepared double layered strontium substituted apatite and titanium dioxide coatings on titanium substrates via mimicking bone mineralization. Morphology, crystallinity, surface chemistry and composition of Sr-substituted coatings formed via biomimetic coating deposition on crystalline titanium oxide substrates were studied as function of incubating temperature and time in phosphate buffer solutions with different Sr ion concentration. The morphology of the biomimetic apatite changed from plate-like for the pure hydroxyapatite to sphere-like for the Sr ion substituted. Surface analysis results showed that 10%-33% of Ca ion in the apatite had been substituted by Sr ion, and that the Sr ions were chemically bonded with apatite and successfully incorporated into the structure of apatite.

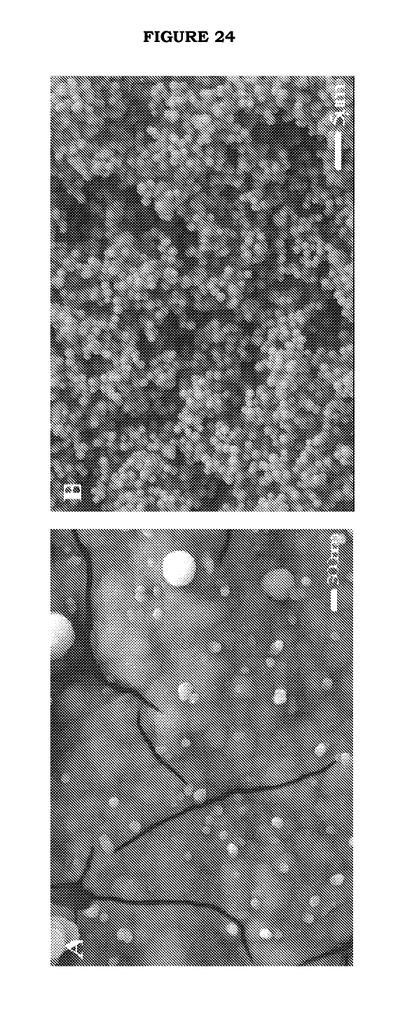

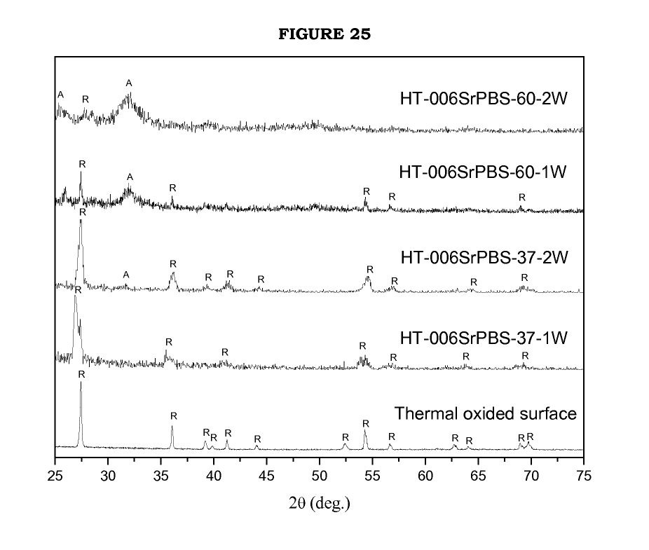

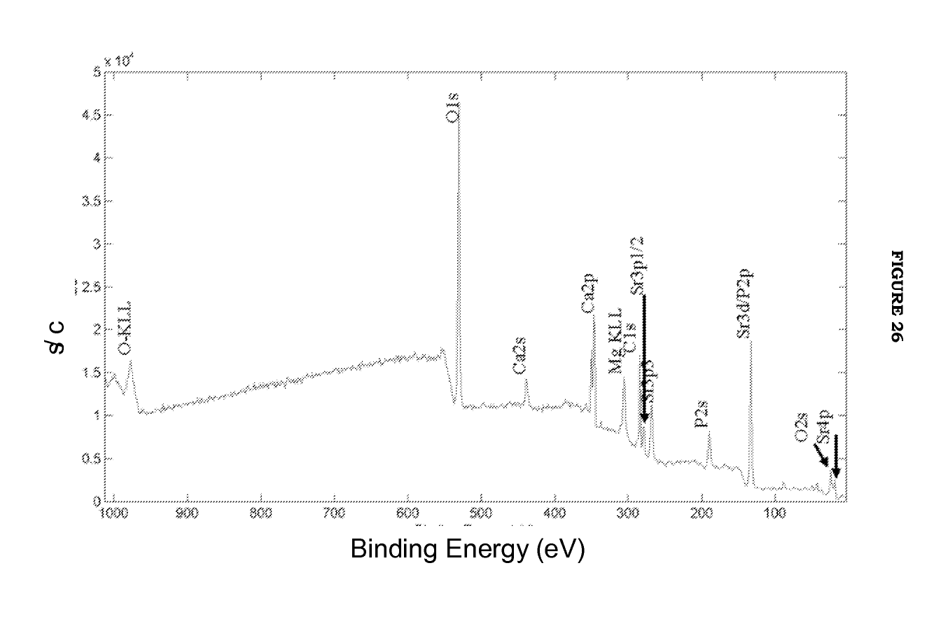

Results are shown in FIGS. 24 to 26.

### Example 7

Fluoride ion substitution on titanium oxide

Sodium fluoride and Dulbecco's phosphate buffered saline (PBS) (D8662, Sigma-Aldrich, USA) was used as incubating medium. The ion composition of the PBS was: Na⁺ (145 mM), K⁺ (4.3mM), Mg²⁺ (0.49 mM), Ca²⁺ (0.91 mM), Cl⁻ (143 mM), H₂PO₄₋ (1.6 mM) and HPO₄²⁻ (8.1 mM). All chemicals were analytical grade regents and used as received without further purification. The obtained phosphate buffer saline was modified by different addition of NaF. Titanium (grade 2, 99.4% pure) was purchased from Edstraco AB (Sweden). Ti plates were treated at 800° C. for 1 hour with a ramping rate of 5° C/min.

This prodecure has been shown to produce a crystalline TiO₂ surface with the rutile structure. After heat treatments the plates were treated in separate ultrasonic baths of alkaline solution (1M NaOH), ethanol and deionized water, separately, in an ultrasonic bath before incubating in the modified phosphate buffer solution.

### Biomimetic Growth of Fluoride Calcium Phosphate

The biomimetic coating was prepared by incubating the pre-treated Ti plates into the phosphate buffered solution with Ca²⁺, H₂PO₄⁻, HPO₄²⁻, and F⁻. The concentration of F ion was 0, 0.04 mM, and 0.2 mM. Ca/P ratio was close to 1/10 in the solution. The pH value was controlled at 7.4 at the beginning. Every titanium plate (10 mm×10 mm×1 mm) was soaked into 20 ml of ion doped phosphate buffered

solution in sealed plastic bottles, kept at 37° C. or 60° C. without stirring for time
periods of 1 day to 2 weeks. For the longer incubating times, the solution was changed every 3 days to avoid depletion of calcium, phosphate and fluoride ions. After immersion, the samples were removed from the solution, rinsed with de-ionized water and allowed to dry in air.

### Characterization

The morphology of the specimens was imaged using field emission scanning electron microscopy (FESEM, LEO 1550). Cross-section images of the coatings were obtained from areas where the coating could be peeled off from the substrate. The crystallinity of the specimens was analyzed using X-ray diffractometry (Siemens Diffractometer 5000) using Cu Kα radiation (λ=1.5418 Å). The diffractometer was operated at 45 kV and 40 mA at a 2θ range of 10°-80° with a fixed incidence angle of 2°. The composition and chemistry of specimens were analyzed by X-ray photoelectron spectroscopy (XPS, Physical Electronics Quantum 2000, Al Kα X-ray source) spectra. XPS survey spectra and high resolution spectra for the F1s peak were acquired.

The morphology of the obtained coatings was needle like and readily deposited on the rutile TiO₂ surface. The diameter of the hydroxyapatite needles was about 10-20 nm, very close to the dimension of minerals in tooth enamel.

### Example 8

Sodium fluoride and Dulbecco's phosphate buffered saline (PBS) (D8662, Sigma-Aldrich, USA) was used as soaking medium. The ion composition of the PBS was: Na⁺ (145 mM), K⁺ (4.3 mM), Mg²⁺ (0.49 mM), Ca²⁺ (0.91 mM), Cl⁻ (143 mM), H₂PO₄₋ (1.6 mM) and HPO₄²⁻ (8.1 mM). All chemicals were analytical grade regents and used as received without further purification.

The morphology of the obtained coatings was needle like and readily deposited on the rutile TiO₂ surface. The rutile coating on the Ti-surface obtained after the heat treatment was rough and contained grains in the micrometer size. Separate bundle of needle-like particles had grown on the surface after the substrate was soaked into the solution containing 0.2 mM F ion for 12 hours at 60° C. After 1 day's soaking, increasing amount of FHA needles grew from the substrate. The diameter of the fluoride substituted hydroxyapatite (FHA) needles was about 10-20 nm, very close to the dimension of minerals in tooth enamel. When the soaking time increased to 1 week, a continuous and homogeneous coating of FHA needles array was formed. It can be seen that the morphology of FHA particles was needle-like and well aligned, unlike the normal flake-like HA crystals formed by the same method using non-modified phosphate buffer solution.

### Example 9

### Surface Ion Release

### Experimental

(1) The initial pH value of PBS and Tris-HCl is 7.40.
(2) Two substrates with ion (Sr, Si, F and Mg) doped calcium phosphate were put into 10 ml solutions in a sealed bottle, and place into an oven at 37° C.
(3) 2 ml of the solution were removed for analysis, and 2 ml fresh solution were added into the bottle at each time point.
(4) The ion concentrations were analyzed by ICP-OES (Inductively coupled plasma optical emission spectroscopy)

A sustained release of the ions was shown and the release rate could be controlled with the ions and the pH. It was also shown that not only release of a single ion is possible but also two.

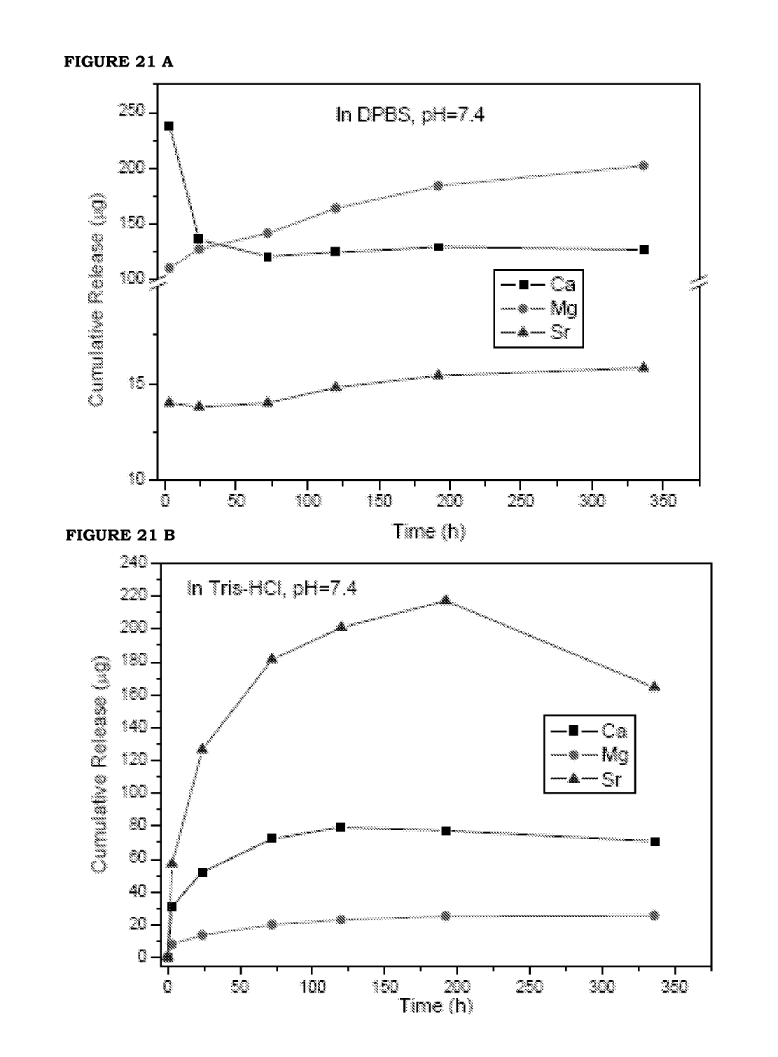

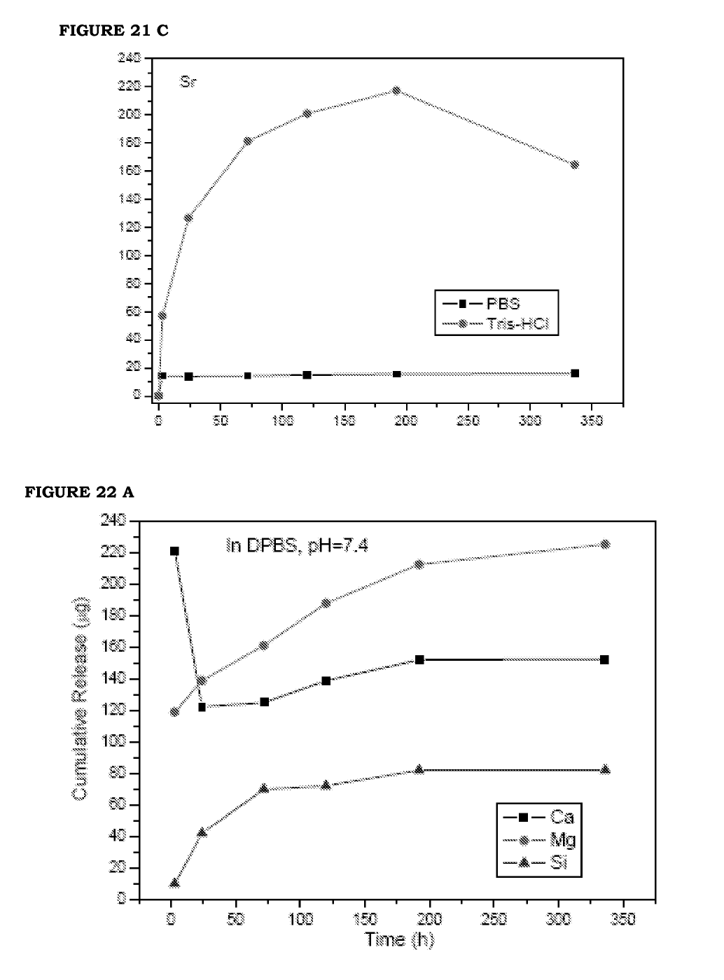

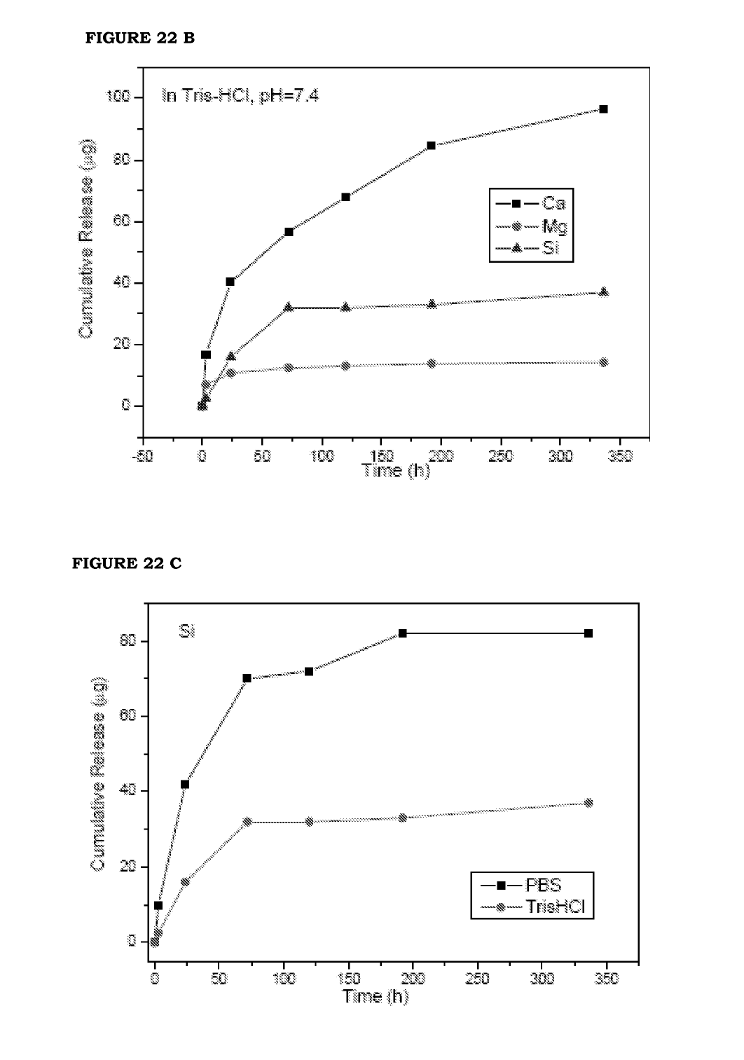

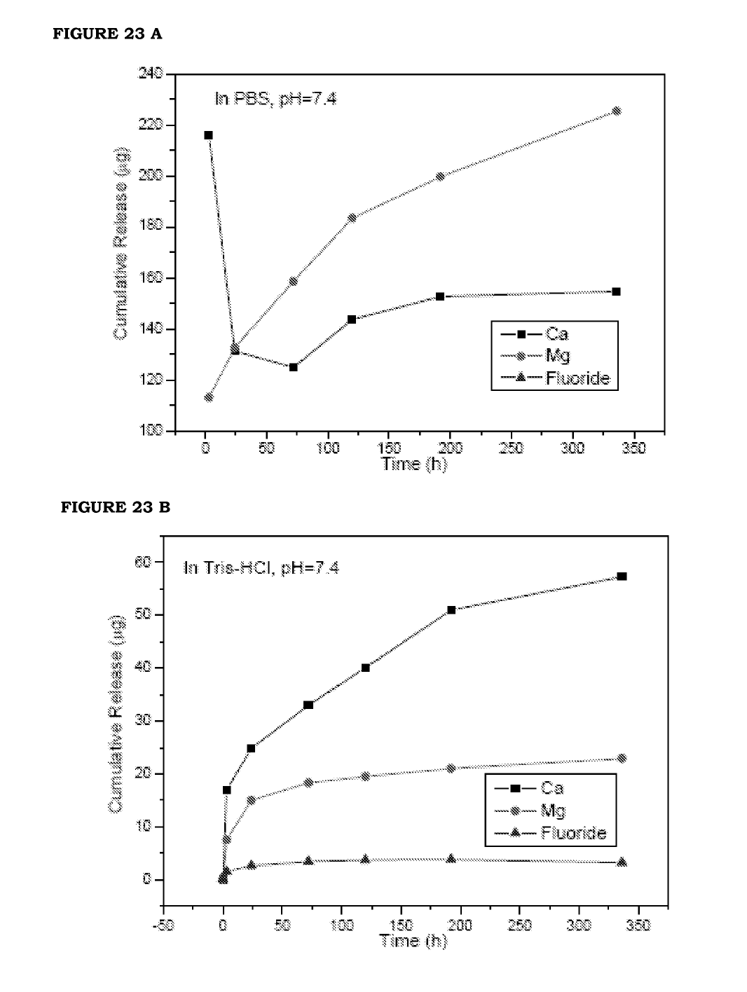

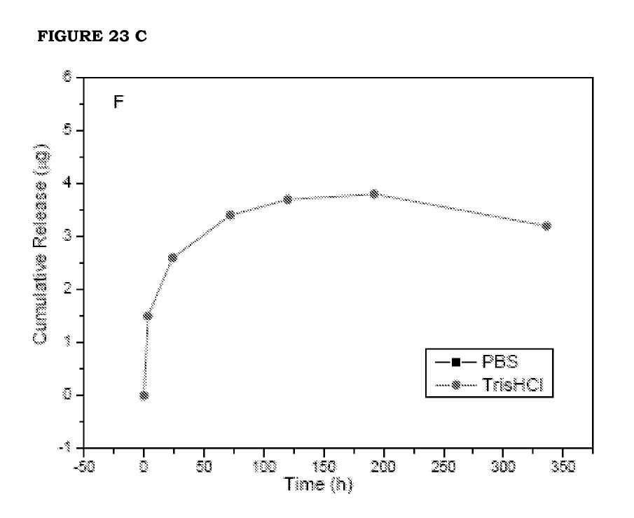

The ion release results are shown in the FIGS. 21 to 23.

### REFERENCES

1. M. Vallet-Regi, J. M. G. Calbet, Calcium phosphate as substitution of bone tissues. Progress in Solid State Chemistry 2004;32:1-31.
2. S. V. Dorozhkin, M. Epple, Bioligical and medical significance of calcium phosphates. Angew Chem Int Ed 2002;41:3130-3146.
3. A. L. Oliveira, R. L. Reis, P. Li, Strontium-substituted apatite coating grown on Ti6A14V substrate through biomimetic synthesis. J Biomed Mater Res Part B 2007;83:258-265.
4. K. A. Gross, L. M. R. Lorenzo, Sintered hydroxyfluorapatites. Part I: Sintering ability of precipitated solid solution powders. Biomaterials 2004;25:1375-1384.
5. C. Robinson, R. C. Shore, S. J. Brookes, S. Strafford, S. R. Wood, J. Kirkham, The Chemistry of Enamel Caries. Crit Rev Oral Biol Med 2000;11:481-495.
6. A. M. Pietak, J. W. Reid, M. J. Stott, M. Sayer, Silicon substitution in the calcium phosphate bioceramics. Biomaterials 2007;28:4023-4032.
7. E. Landi, A. Tampieri, G. Celotti, S. Sprio, M. Sandri, G. Logroscino, Sr-substituted hydroxyapatites for osteoporotic bone replacement. Acta Biomaterials 2007;3:961-969.
8. E. Landi, A. Tampieri, M.M. Belmonte, G. Celotti, M. Sandri, A. Gigante, P. Fava, G. Biagini, Biomimetic Mg- and Mg,CO₃-substituted hydroxyapatites: synthesis charcterization and in vitro behaviour. J Euro Ceram Soc 2006;26:2593-2601.
9. W. Xue, H. L. Hosick, A. Bandyopadhyay, S. Bose, C. Ding, K. D. K. Luk, K. M. C. Cheung, W. W. Lue, Preparation and cell-materials interactions of plasma sprayed strontium-containing hydroxyapatite coating. Surface 85 Coatings Technology 2007;201:4685-4693.
10. G. Qi, S. Zhang, K. A. Khor a, W. Weng, X. Zeng, C. Liu, An interfacial study of sol-gel-derived magnesium apatite coatings on Ti6Al4V substrates. Thin Solid Films 2008;516:5172-5175.
11. E. S. Thian, J. Huang, S. M. Best, Z. H. Barber, W. Bonfield, Novel Silicon-Doped Hydroxyapatite (Si-hydroxyapatite) for Biomedical Coatings: An In Vitro Study Using Acellular Simulated Body Fluid. J Biomed Mater Res Part B: Appl Biomater 2006;76:326-333.
12. E. L. Solla, P. Gonzalez, J. Serra, S. Chiussi, B. Leon, J. Garcia Lopez, Pulsed laser deposition of silicon substituted hydroxyapatite coatings from synthetical and biological sources. Applied Surface Science 2007;254:1189-1193.
13. Y. Han, D. H. Chen, L. Zhang, Nanocrystallized Srhydroxyapatite/Srhydroxyapatite-SrTiO₃/SrTiO₃-TiO₂ multilayer coatings formed by micro-arc oxidation for photocatalytic application. Nanotechnology 2008;19:335705.
14. B. C. Bunker, P. C. Rieke, B. J. Tarasevich, A. A. Campbell, G. E. Fryxell, G. L. Graff, L. Song, J. Liu, J. W. Virden, G. L. McVay, Ceramic Thin-Film Formation on Functionalized Interfaces Through Biomimetic Processing Science 1994;264:48-55.
15. Li P. Patent: Strontium substituted apatite coating. U.S. Pat. No. 6,905,723 B2 2005.
16. Li P. Patent: Bioactive ceramic coating and method. U.S. Pat. No. 6,569,489 B1 2003.
17. X. Zhao, X. Liu, C. Ding, Acid-induced bioactive titania surface. J Biomed Mater Res A 2005;75:888-894.
18. C. Wu, J. Chang, W. Zhai, S. Ni, A novel bioactive porous bredigite (Ca₇MgSi₄O₁₆) scaffold with biomimetic apatite layer for bone tissue engineering. J Mater Sci-Mater in Med 2007;18(5):857-864.

1. A method for forming a surface coating of an ion substituted calcium phosphate with controlled morphology on a substrate comprising the steps:
a. providing said substrate;
b. pre-treating said substrate in order to create an activated surface;
c. providing an aqueous solution comprising calcium ions, magnesium ions, phosphate ions and one or more of substitution ions selected from Sr²⁺, Si⁴⁺, F⁻, Ba²⁺, Fe³ and Zn²⁺, and optionally further comprising one or more of ions selected from sodium ions, potassium ions, chloride ions, carbonate ions and sulfate ions, having an initial pH in the range of 2.0 to 10.0 and a temperature of 20° C. to 100° C.; and
d. incubating at least a portion of the substrate in the aqueous solution for a period of time sufficient for the coating to be formed.

2. The method according to claim 1 wherein the method for forming a surface coating of an ion substituted calcium phosphate with controlled morphology on a substrate comprises the steps:
a. providing a substrate;
b. pre-treating said substrate in order to create an activated surface;
c. providing an aqueous solution comprising calcium ions, magnesium ions, phosphate ions and one or more of substitution ions selected from Sr²⁺, Si⁴⁺, F⁻, Ba²⁺, Fe³ and Zn²⁺, and optionally further comprising one or more of ions selected from sodium ions, potassium ions, chloride ions, carbonate ions and sulfate ions, having an initial pH in the range of 6.0 to 8.0 and a temperature of 20° C. to 100° C.;
d. incubating at least a portion of the substrate in the first aqueous solution for a period of time sufficient for a first coating to be formed;
e. providing a second aqueous solution comprising calcium ions, magnesium ions, phosphate ions and one or more of substitution ions selected from Sr²⁺, Si⁴⁺, F⁻, Ba²⁺, Fe³ and Zn²⁺, and optionally further comprising one or more of ions selected from sodium ions, potassium ions, chloride ions, carbonate ions and sulfate ions, wherein the solution has an initial pH in the range of 6.0 to 8.0 and a temperature of 20° C. to 100° C.; and
f. incubating at least a portion of the substrate in the second aqueous solution for a period of time sufficient for a second layer of coating to be formed.

3. The method according to claim 2 wherein the steps c) to f) are repeated in order to create additional layers optionally containing another coating chemistry and morphology.

4. The method according to claim 1 wherein the pre-treating involves formation of a calcium phosphate layer.

5. The method according to claim 1 wherein the pre-treating involves heat treatment, hydrolysis, oxidation, acid or base treatment, anodic oxidation, UV radiation, CVD, sol-gel or PVD.

6. The method according to claim 4 wherein the pre-treatment results in formation of charged groups on the substrate surface.

7. The method according to claim 1 wherein the immersion time in each solution is up to 2 weeks, preferably less then 1 week and more preferably less than 3 days.

8. The method according to claim 1 wherein the concentration of:
calcium ions is in the range 0.01-25 10⁻³M, preferably 0.5-2.5 10⁻³M;
magnesium ions is in the range 0.01-15 10⁻³M, preferably 0.2-1.5 10⁻³M;
sodium ions is in the range 0.01-1420 10⁻³M, preferably 100-150 10⁻³M;
potassium ions is in the range 0.01-1420 10⁻³M, preferably 1.0-5.0 10⁻³M;
chloride ions is in the range 0.01-1030 10⁻³M, preferably 100-150 10⁻³M;
phosphate ions is in the range 0.01-10 10⁻³M, preferably 1.0-10 10⁻³M;
carbonate ions is in the range 0.01-270 10⁻³M, preferably 1.0-50 10⁻³M;
sulfate ions is in the range 0.01-5 10⁻³M, preferably 0.1-1.0 10-3M.

9. An ion substituted calcium phosphate surface coating comprising calcium, magnesium, phosphate and one or more of strontium, silicon, fluoride, barium, iron and zinc and optionally one or more of sodium, potassium, chloride, carbonate and sulfate.

10. The surface coating according to claim 9 wherein the cationic substitution of calcium is up to 80%, preferably 25-60%.

11. The surface coating according to claim 9 wherein the anionic substitution of phosphate and hydroxide is up to 30%, preferably 10-25%.

12. The surface coating according to claim 9 wherein the coating contains 0-5%, preferably 1.5-3% of fluoride, or 0-10%, preferably 3-8% strontium, or 0-5%, preferably 0.5-2% silicon, or combinations thereof.

13. The surface coating according to claim 9 wherein the morphology of the coating is in the form of sheets, flakes, spheres, porous structures, spikes or rods or a combination thereof.

14. The surface coating according to claim 9 wherein the coating comprises multiple layers.

15. The surface coating according to claim 9 wherein the coating is bioresorbable.

16. Use of an ion substituted calcium phosphate coating according to claim 7 as a drug and/or ion delivery system.
